(19) United States
(12) Patent Application Publication
(10) Pub. No.: US 2012/0088679 A1
(43) Pub. Date: Apr. 12, 2012

(54) Method for Monitoring Cell Culture

(75) Inventors: Chetan Goudar, Milpitas, CA (US); Maria Klapa, Piraeus (GR)

(73) Assignee: BAYER HEALTHCARE LLC, Tarrytown, NY (US)

(21) Appl. No.: 13/255,877

(22) PCT Filed: Mar. 10, 2010
(86) PCT No.: PCT/US2010/026843

§ 371 Date: Dec. 13, 2011

Related U.S. Application Data
(60) Provisional application No. 61/158,954, filed on Mar. 10, 2009.

(51) Int. Cl. C40B 30/00; C12Q 1/02

(52) U.S. Cl. 506/7; 435/29

(57) ABSTRACT

The present invention is directed to a method of monitoring the physiological state of a cell cultivation. Several parameters such as cell viability, growth, metabolic profile, and productivity may be monitored to establish a metabolic fingerprint or metabolomic profile of a cell culture.

This application claims the benefit of U.S. Provisional Application Ser. No. 61/158,954; filed on Mar. 10, 2009, the contents of which are incorporated herein by reference in their entirety.

### FIELD OF THE INVENTION

The present invention is directed to a method of monitoring the physiological state of a cell cultivation. Several parameters such as cell viability, growth, metabolic profile, and productivity may be monitored to establish a metabolic fingerprint or metabolomic profile of a cell culture.

### BACKGROUND OF THE INVENTION

Mammalian cell cultures have been widely used for the production of therapeutic proteins, in which complex post translational modifications are necessary to ensure efficacy in patients. To-date, large-scale fed-batch cultivation remains the dominant mode of therapeutic protein production (Chu, et al., Curr. Opin. Biotechnol. 12:180-187, 2001). High-density perfusion cultivation (Konstantinov, et al., Adv. Biochem. Eng. Biotechnol. 101:75-98, 2006; Konstantinov, et al., Biotechnol. Prog. 12:100-109, 1996; Trampler, et al., Biotechnology 12:281-284,1994) is typically employed in the case of unstable molecules, for which minimal residence times at elevated bioreactor temperatures are desirable (see, e.g., FIG. 1). In light of a) the dosing demand and the pricing pressure, b) the strict protein quality requirements of regulatory agencies, and c) the aggressive timelines for industrial process development, the primary objective of most current process development programs for therapeutic protein production is the rapid development of bioreactor processes that are characterized by high product yield and consistent product quality. In addition, due to the high manufacturing cost of these processes, the identification and use of accurate and sensitive controls for cell cultivation robustness is desirable. These controls could provide early warnings of problems in protein productivity and/or final quality before the end of the cultivation. Currently, both bioreactor monitoring and process improvements are based primarily on cell growth, metabolic activity, and protein productivity data. While useful, the limitations of this cell specific rate-based approach have been recognized and approaches such as quasi real-time metabolic flux analysis have been suggested for more robust characterization of the cellular physiological state (Goudar, et al., Adv. Biochem. Eng. Biotechnol. 101:99-118, 2006; Konstantinov, Biotechnol. Bioeng. 52:271-289, 1996).

There is thus a clear need for the development and application of methods that enable the comprehensive characterization of the physiological state of mammalian cell cultures, improving over the current conventional set of measurements for monitoring process consistency and robustness. Moreover, utilized in the context of continuous process improvement programs that allow for experimentation with various cell lines and experimental conditions, these methods could generate large physiological datasets, which would enhance overall understanding of the protein production and manufacturing processes. Such developments could lead to the identification of accurate and sensitive markers not only of protein productivity, but of protein quality too. These markers could be subsequently used in the manufacturing process for the prediction of the final protein quality before the end of the cultivation, while they could also help in improving the protein production and manufacturing process to ensure batch to batch consistency in the final result. Finally, these physiological characterization methods could be based on a cost-efficient platform.

To address the characterization of the physiological state of mammalian cell cultures, gas chromatography-mass spectrometry (GC-MS) metabolomics was utilized to analyze baby hamster kidney (BHK) cells cultivated in high-cell density perfusion reactors at both laboratory and manufacturing scales. Metabolomic profiling enabled the differentiation of cell cultures based on cell age, bioreactor scale and cell source, while the identification of the differentiating metabolites provided important information about the in vivo physiological state of the cultures. As such, metabolomics is a valuable molecular analysis tool in cell culture engineering.

### DESCRIPTION OF THE FIGURES

FIG. 1: Schematic of the cell culture perfusion system.

FIG. 2: Overview of the fermentation process. The lines connecting vials and reactors indicate the inoculation source for the laboratory and manufacturing scale bioreactors.

FIG. 3: The time profile of viable cell density for reactors over the entire course of operation.

FIG. 4: The time profiles of a) bioreactor viability, b) cell growth, c) specific glucose consumption rate and d) specific lactate production rate for the reactors. The course of the cultivation for a reactor was divided into 10-days intervals starting from the attainment of steady-state and the average value for each interval along with the associated standard deviation is presented at the middle time-point of the interval.

FIG. 5: 5A. Hierarchical Clustering (HCL) and 5B. Principal Component Analysis (PCA) of the GC-MS polar metabolic profiles of the laboratory-scale bioreactors. Both analyses were based on the standardized relative peak areas for each metabolite in the profiles, as defined in Equation (1). Pearson correlation was the distance metric for HCL. In FIG. 5A, the centroid graph of the profiles included in each of the 3 identified sub-clusters is also depicted. PC1, PC2, and PC3 refer to the % variation from the dataset in the original experimental space that is carried by principal components 1, 2, and 3, respectively.

FIG. 6: 6A. Hierarchical Clustering (HCL) and 6B. Principal Component Analysis (PCA) of the GC-MS polar metabolic profiles of the manufacturing-scale bioreactors. Both analyses were based on the standardized relative peak areas for each metabolite in the profiles, as defined in Equation (1). Euclidean was the distance metric for HCL. In FIG. 6A, the centroid graph of the profiles included in each of the 2 identified sub-clusters is also depicted. PC1, PC2, and PC3 refer to the % variation from the dataset in the original experimental space that is carried by principal components 1, 2, and 3, respectively.

FIG. 7: 7A. Hierarchical Clustering (HCL) with Manhattan distance metric, 7B. Hierarchical Clustering with Kendal-Tau distance metric, 7C. Hierarchical Clustering with Spearmn Correlation distance metric, and 7D. Principal Component Analysis (PCA) of the GC-laboratory-scale MS polar metabolic profiles of the manufacturing-scale bioreactors M1 and M2 and the bioreactors L2 and L3. All analyses were based on the standardized relative peak areas for each metabolite in the profiles, as defined in Equation (1) in the text. All depicted symbols are used as explained in the legends of FIGS. 5 and 6.

FIG. 8: The metabolites whose concentration was identified as significantly increased in the 129 day sample compared to the 122 day for reactor M1 sample in the context of the metabolic network (in bold boxes). The significant metabolites were identified using Significant Analysis for Microarrays (SAM) for δ=1.64 and 0% FDR (median). The metabolites that were included in the analysis after normalization and filtering, but were not identified as significant by SAM, are shown in blank boxes.

### DESCRIPTION OF THE INVENTION

It is to be understood that this invention is not limited to the particular methodology, protocols, cell lines, animal species or genera, constructs, and reagents described and as such may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a,” “and,” and “the” include plural reference unless the context clearly dictates otherwise. Thus, for example, reference to “a metabolite” is a reference to one or more metabolites (e.g., one, two, five, ten, fifty, one hundred, or more) and includes equivalents thereof known to those skilled in the art, and so forth.

Unless defined otherwise, all technical and scientific terms used herein have the same meaning as commonly understood to one of ordinary skill in the art to which this invention belongs. Although any methods, devices, and materials similar or equivalent to those described herein can be used in the practice or testing of the invention, the preferred methods, devices and materials are now described.

All publications and patents mentioned herein are hereby incorporated herein by reference for the purpose of describing and disclosing, for example, the constructs and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

Cell culture engineering has to-date used transcriptomic, proteomic, and metabolic flux analyses, attempting to resolve significant questions regarding cell culture performance. However, there is a clear need for the development and application of methods that enable the comprehensive characterization of the physiological state of mammalian cell cultures, improving over the current conventional set of measurements for monitoring process consistency and robustness.

The high-throughput molecular analysis platform of metabolomics could satisfy this need. Metabolomics, referring to the simultaneous quantification of the (relative) concentration of the free small metabolite pools, enables the monitoring of a metabolic fingerprint of a biological system (Fiehn, et al., Nat. Biotechnol. 18:1157-1168, 2000; Roessner, et al., Plant J. 23:131-142, 2000). Considering the role of metabolism in the context of overall cellular function, it is easily understandable why quantifying a complete and accurate metabolic profile map could be of great importance in cell culture engineering research. While metabolite concentrations and metabolic fluxes are not linearly related, metabolic profiling is high-throughput and can thus be easily used to monitor transient metabolic conditions. In addition, no knowledge of the structure and regulation of the investigated metabolic networks is needed as is the case with metabolic flux analysis (MFA). In addition, MFA is typically applied only to steady-state or pseudo-steady-state conditions while metabolomics can be used under transient physiological conditions like transcriptomics and proteomics, the other two main omic platforms. Also, unlike transcriptomics and proteomics, metabolomics does not require special analytical equipment. Metabolomic methodologies are based on classical analytical chemistry techniques, including mainly the nuclear magnetic resonance (NMR) spectroscopy and mass spectrometry (MS) (e.g., gas chromatography-mass spectrometry and liquid chromatography-mass spectrometry), and are the least costly of the omics approaches (Kanani, et al., J. Chromatogr B. Analyt. Technol. Biomed. Life Sci. 871:191-201, 2008). For cell culture systems in particular, a special advantage of metabolomics over the other omics technologies is its applicability to monitor both the intracellular metabolic state and the composition of the extracellular medium. This provides a better understanding of the metabolic network activity. Finally, as metabolism is well conserved among biological systems, comparative metabolomic studies are easier and do not require sophisticated normalization among samples.

Monitoring in vivo metabolic physiology through molecular fingerprinting allows determination of subtle differences between samples that may not be directly observable with conventional measurements. Enhanced characterization of cell physiological state is possible by metabolic fingerprinting which can improve currently used process control methodologies. Moreover, multivariate statistical analysis enables metabolite identification whose concentration change characterizes the difference in the cell culture physiology. This information can help identify early warning markers for inconsistent performance and can also further the understanding of cell physiology eventually leading to process optimization.

Metabolomic fingerprinting could be a very useful molecular analysis tool in cell culture engineering. Individually or integrated with other high-throughput molecular analysis techniques that assess other levels of cellular function could provide leads towards the optimization of the fermentation process and the enhancement of the currently available measurement set that is used to monitor the status and the quality consistency of the fermentation campaign.

The present invention relates to the use of GC-MS metabolomics of cell cultures to monitor mammalian cell physiology in high density perfusion cultures. GC-MS metabolomics may be used to analyze the cellular physiological state at different cultivation stages for both laboratory and manufacturing scales.

### EXAMPLES

In order that this invention may be better understood, the following examples are set forth. These examples are for the purpose of illustration only, and are not to be construed as limiting the scope of the invention in any manner. All publications mentioned herein are incorporated by reference in their entirety.

### Example 1. Cell Perfusion Cultivation

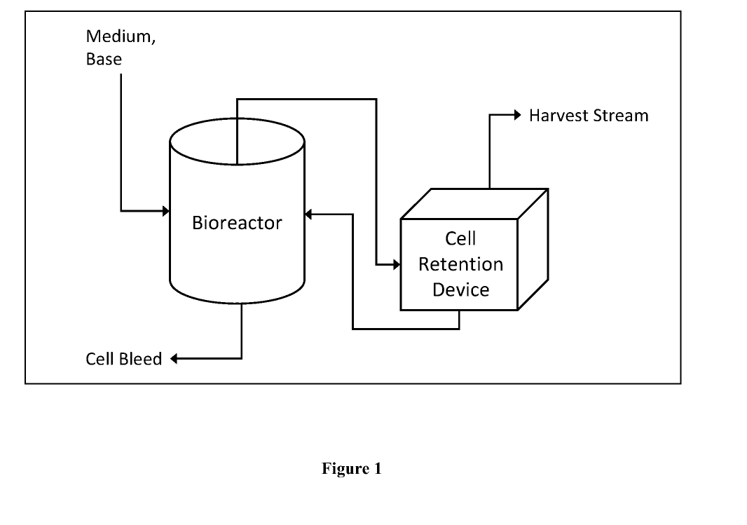

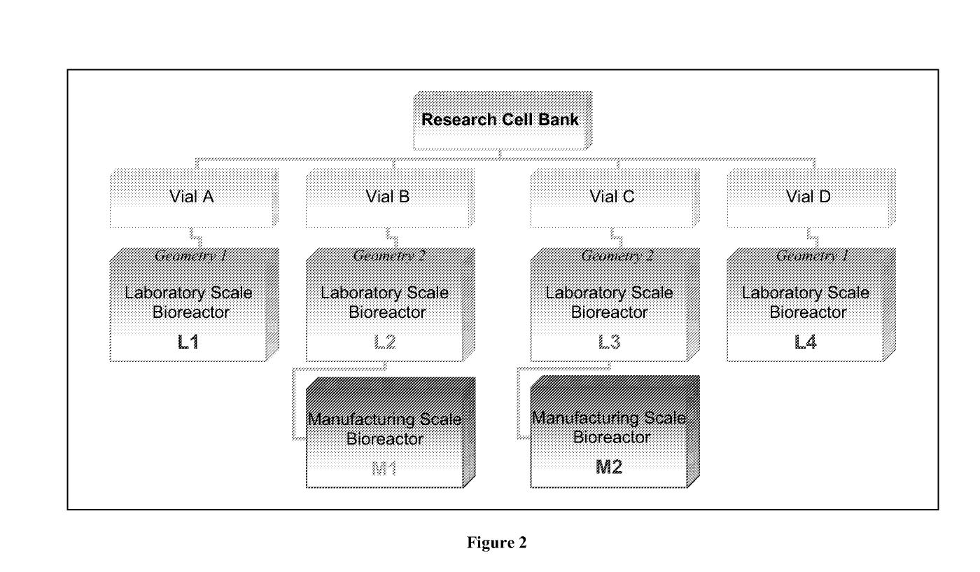

BHK cells were cultivated in perfusion mode (FIG. 1) with glucose and glutamine as the main carbon sources. Four independent vials of baby hamster kidney (BHK) cells (A-D in FIG. 2) were used to inoculate four laboratory-scale 15L perfusion systems, L1-L4, respectively. The L1 and L4 bioreactors were typical cylindrical vessels, while L2 and L3 were small-bottom reactors. L2 and L3 were used to inoculate the two manufacturing-scale bioreactors M1 and M2, respectively. All laboratory- and manufacturing-scale perfusion systems had the same operating conditions and set-points for all monitored variables. The duration of the bioreactor runs ranged from 113-155 days resulting in a combined total of 826 bioreactor days. During the last 40 days of operation of each reactor, samples were collected at various time points, as shown in Table 1. Cell age is measured from the date of vial thaw and cultivation time in same reactor is estimated as the time difference between the start date of each reactor and the date of sample collection. The cell pellets from these samples were analyzed using GC-MS metabolomics and multivariate statistical analysis.

TABLE 1

| | Reactor | Reactor | Cell Age | Cultivation Time in |
|---|---|---|---|---|
| Sample # | Name | Scale | (days) | Same Reactor (days) |
| 1 | L4 | Laboratory | 95 | 95 |
| 2 | | | 123 | 123 |
| 3 | L1 | | 120 | 120 |
| 4 | | | 148 | 148 |
| 5 | L2 | | 122 | 122 |
| 6 | | | 150 | 150 |
| 7 | L3 | | 121 | 121 |
| 8 | M1 | Manufacturing | 122 | 85 |
| 9 | | | 129 | 92 |
| 10 | | | 150 | 113 |
| 11 | M2 | | 128 | 100 |
| 12 | | | 149 | 128 |

The bioreactor temperature was maintained at 35.5° C. and the agitation at 47 RPM (15 RPM for manufacturing-scale bioreactors). The dissolved oxygen (DO) concentration was maintained at 50% air saturation by membrane aeration, and the pH was maintained at 6.8 by automatic addition of 6% Na₂CO₃. The bioreactors were inoculated at an initial cell density of ˜1×10⁶ cells/mL and the cells were allowed to accumulate to a steady-state concentration of 20×10⁶ cells/mL. This target steady-state cell density was maintained by automatic cell bleed from the bioreactor.

Samples were collected daily from each bioreactor for cell density and viability analyses using the CEDEX system (Innovatis, Bielefeld, Germany). These samples were subsequently centrifuged (Beckman Coulter, Fullerton, Calif.) and the supernatants were analyzed for nutrient and metabolite concentrations. Glucose, lactate, glutamine, and glutamate concentrations were determined using a YSI model 2700 analyzer (Yellow Sprints Instruments, Yellow Springs, Ohio), while ammonia was measured by an Ektachem DT60 analyzer (Eastman Kodak, Rochester, N.Y.). The pH and DO were measured online using retractable electrodes (Metler-Toledo Inc., Columbus, Ohio) and their measurement accuracy was verified through off-line analysis in a Rapidlab 248 blood gas analyzer (Bayer HealthCare, Tarrytown, N.Y.). The same instrument was used to also measure the dissolved CO2 concentration. On-line measurements of cell density were made with a retractable optical density probe (Aquasant Messtechnik, Bubendorf, Switzerland), calibrated with cell density measurements from the CEDEX system.

### Example 2: Metabolomic Profiling

Samples from the bioreactor were drawn on ice and centrifuged using a pre-cooled rotor. Following centrifugation, the supernatant was discarded and the cell pellet was washed with cold PBS buffer. The cell pellet was then placed for 15 minutes in 70° C. water bath. The pellet was subsequently dried in vacuum at 70° C. for 24 hours. The dried cell pellets from samples were used for metabolomic analysis.

The polar metabolite extracts of the dried cell pellets were obtained using methanol/water extraction (Kanani, et al., 2008; Roessner, et al., 2000) with ribitol (0.1 mg/g of dry cell weight) and [U-13C]-glucose (0.2 mg/g of dry cell weight) as internal standards. The dried polar extracts were derivatized to their (MeOx)TMS-derivatives through reaction with 150 μL methoxyamine hydrochloride solution (20 mg/mL) in pyridine for 90 minutes, followed by reaction with 300 μL N-methyl-trimethylsilyl-trifluoroacetamide (MSTFA) for at least 6 hours at room temperature (Kanani, et al., 2008; Kanani, et al., Metab. Eng., 9:39-51, 2007). The metabolomic profiles were obtained using the Saturn 2200T Gas Chromatograph—(ion trap) Mass Spectrometer (Varian Inc., Calif.). The peak identification and quantification was carried out as described in (Kanani, et al., 2007). The raw metabolomic dataset comprised of 91 peaks, each of which was detected in at least one of the acquired metabolomic profiles and corresponds to a compound of known chemical category (see, e.g., Kanani, et al., 2008; Kanani, et al., 2007).

The relative areas of all detected peaks (RPAs) were estimated from normalization with ribitol (marker ion: 217), the internal standard. Data validation, normalization, and correction methodology was applied to account for the derivatization biases that are primarily due to the formation of multiple derivatives from the amine-group containing metabolites (Kanani, et al., 2008; Kanani, et al., 2007). GC-MS operating conditions were verified during the acquisition of acquired metabolomic profiles of samples based on the ratio of the two peaks of [U-13C]-glucose. Secondly, the derivative peak areas that corresponded to the same amine-group containing metabolite were combined into one cumulative (effective) peak area, using the weight coefficients that were estimated based on the amino acid derivatives' profiles of the 95 day L4 sample (see Table 1). Isoleucine, β-alanine, and gluatamate were filtered out of further analysis, because their available measurements did not allow for all positive weight coefficients to be estimated. The amino acids, for which only one derivative was observed in the particular derivatization range, but for which more than one derivative are known, were included in the subsequent step of the analysis; most often they were filtered out at the latter step, because of high coefficient of variation between injections. Further, (a) the smallest of the two MeOx peaks of the known ketone-group containing metabolites, (b) the peaks corresponding to unknown amine-group containing metabolites, (c) the peaks that were identified as derivatization artifacts or with significant carry over, and (d) the metabolite peaks that were not consistently detected were filtered out of the analysis. These final metabolite RPA profiles, comprising 38 metabolites, were used in the TM4 MeV (V4.0) data analysis software (Saeed, et al., Biotechniques 34:374-378, 2003) with 80% cut-off. Any missing RPAs were imputed using the k-nearest neighbor's algorithm (Troyanskaya, et al., Bioinformatics 17:520-525, 2001) as implemented in TM4 MeV.

Analyses applied on the acquired metabolic profiles were based on the standardized values of the metabolite relative peak areas (see Equation 1 below). The use of standardized relative peak areas in these analyses is required due to the large variance in the order of magnitude of the relative peak area levels between metabolites in the same metabolic profile. The standardized relative peak area of metabolite M in the metabolomic profile j, RPAMj, is equal to:

$${RPA}_{{M}_{j}} = \frac{{RPA}_{{M}_{j}} - mean {RPA}_{M} ( over all profiles )}{{SD}_{{RPA}_{M}} ( over all profiles )} \qquad ( 1 )$$

Hierarchical Clustering (HCL) was used to cluster the samples based on their metabolomic profiles. In HCL, the metabolic profiles are clustered in a hierarchical tree. At the lowest level of the tree, each metabolic profile is considered as a separate cluster, while all samples are grouped in one cluster at the highest level. Starting from the lowest level, a correlation coefficient for each pair of the available clusters is estimated based on a particular distance metric at each round of the algorithm. Clusters with the highest correlation coefficient are grouped into one cluster for the subsequent round of the algorithm (Quackenbush, Nat. Genet. 2:418-427, 2001; Eisen, et. al., Proc. Natl. Acad. Sci. USA 95:14863-14868, 1998). The acquired hierarchical tree has to be interpreted in the context of the biological problem being studied. For example, in FIG. 5A, HCL identifies two clusters of metabolic profiles that correspond to cell ages a month apart. Within the lower cell age cluster, the metabolic profile of reactor L4 separates from the metabolic profiles of the other three laboratory-scale reactors.

Principal Component Analysis (PCA) was used to visualize whether the various cell culture samples could be differentiated based on their metabolomic profiles. PCA involves the singular value decomposition of a data matrix, which refers to the orthogonal linear transformation of the original dataset on a new coordinate system that captures the maximum variance in the dataset (Strang, Introduction to Linear Algebra, Wellesley-Cambridge Press, Wellesley, Mass., 1993). This transformation may involve rotating and/or stretching of the original experimental space. Principal component 1 corresponds to the direction of the highest variance in the original dataset, principal component 2 the second highest and so on. In high-throughput biological data analysis, PCA has been used for coordinate reduction purposes, so that a majority of the variance in the original dataset is visualized within the 3-D space (Raychaudhuri, et. al., Pac. Symp. Biocomput. 2000:455,466, 2000). A small number of principal components is often sufficient to account for most of the structure in the data (Scholkopf, et al., Learning with Kernels—Support Vector Machines, Regularization, Optimization and Beyond, The MIT Press, Cambridge, Mass., 2002). When reading a PCA graph, it is important to consider the percentage of the variance in the original dataset that is captured within the indicated space in total and by each of the shown principal components individually. The unit (weight) of each principal component is equal to the percentage of variance in the original dataset that is represented by it. Thus, for example, two data points with distance x on principal component 1 represent larger difference in their physiological state than two data points with the same distance on principal component 2.

The metabolites, whose concentration was significantly higher or lower in one set of cell culture samples compared to another set was referred to as positively or negatively, respectively, significant metabolites of the particular comparison. The significant metabolites in one comparison were identified using the unpaired Significance Analysis of Microarrays (SAM) approach (Tusher, et al., Proc. Natl. Acad. Sci. USA 98:5116-5121, 2001).

SAM (Tusher, et al., 2001; Larsson, et al., BMC Bioinformatics 6:129, 2005; Wu, Bioinformatics 21:1565-1571, 2005) is a permutation-based (non-parametric) hypothesis testing method for the identification of molecular quantities that differ significantly between two measurement sets that represent different physiological conditions. SAM has been tailored for the analysis of transcriptional profiling data based on DNA microarrays and has similarly been used for the analysis of other omic datasets (see, e.g., Dutta, et. al., Biotechnol. Bioeng. 102:264-279, 2009). In the case of the metabolomic analysis, SAM identifies metabolites whose difference in concentration between two samples is larger than the difference that would have been anticipated due to random variations alone:

═dᵢ−deᵢ>δ

where dᵢ is the observed difference, deᵢ the expected difference, and δ the significance threshold. Unlike parametric hypothesis testing methods, permutation-based (non-parametric) methods do not require the data to follow a particular distribution. They also provide an estimation of the false discovery rate (FDR) which is the probability that a given metabolite identified as differentially changing in concentration is a false positive. In addition, SAM allows δ adjustment such that the sensitivity of the FDR and number of significant metabolites to the threshold change can be determined.

Algorithms were used as implemented in TM4 MeV v4.0 (Saeed et al., 2003). A holistic view of the difference in metabolic profile between two cell culture samples was obtained by positioning the identified as significant metabolites in the appropriately color-coded metabolic network. The metabolic network reconstruction was based on information from the KEGG (KEGG Database, 2008) and EXPASY (EXPASY Database, 2008) databases.

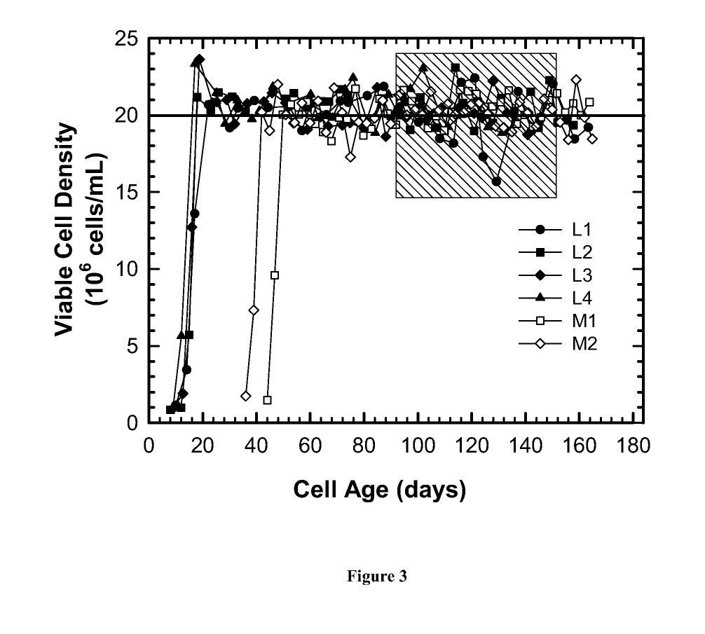

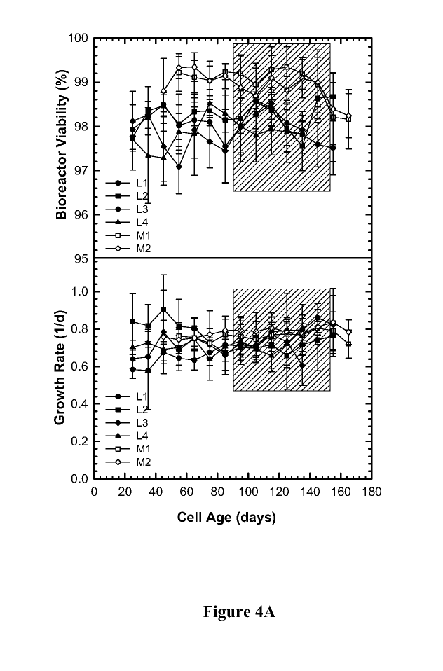

One of the key variable that needs to be carefully controlled in a perfusion system to ensure the consistency of the process is the viable cell density. It is typically maintained at the target set-point throughout the course of the cultivation after the perfusion system has reached steady-state conditions. The steady-state cell density remained close to the target set point of 20×10⁶ cells/mL (Table 2, FIG. 3). FIG. 3 depicts the time profile of viable cell density for the reactors over the course of their operation, while the steady-state averages are shown in Table 2. Steady-state averages for cell growth-, metabolic activity-, and productivity-related variables are shown. The coefficient of variation is shown in parenthesis. FVCD, sGCR, and sLPR are the bioreactor viable cell density, specific glucose consumption rate, and specific lactate production rate, respectively. The average specific productivity of all reactors are shown relatively to the average value for the L1 reactor.

It has been shown that cell density measurements are associated with ˜8.5% error which suggests that that the observed variation around the mean values (i.e., coefficients of variation (CoV's) in the range of 4.4% - 6.3%) is very small reflective of good cell density control.

TABLE 2

| | L1 | L2 | L3 | L4 | M1 | M2 |
|---|---|---|---|---|---|---|
| | (154 d) | (155 d) | (154 d) | (129 d) | (113 d) | (121 d) |
| BVCD | 19.89 | 20.48 | 20.18 | 20.68 | 20.23 | 20.12 |
| (10⁶ cells/mL) | (6.3%) | (5.0%) | (6.2%) | (5.3%) | (4.4%) | (4.7%) |
| Viability | 98.0 | 98.2 | 97.9 | 97.8 | 99.0 | 98.9 |
| (%) | (0.6%) | (0.6%) | (0.7%) | (0.7%) | (0.6%) | (0.6%) |
| Growth Rate | 0.72 | 0.75 | 0.70 | 0.71 | 0.76 | 0.78 |
| (1/d) | (17.9%) | (17.2%) | (15.9%) | (12.1%) | (10.0%) | (12.1%) |
| sGCR | 1.59 | 1.76 | 1.62 | 1.65 | 1.53 | 1.54 |
| (pmol/cell-d) | (14.7%) | (12.0%) | (12.6%) | (11.3%) | (9.3%) | (9.9%) |
| sLPR | 2.04 | 2.21 | 2.13 | 2.14 | 2.03 | 2.06 |
| (pmol/cell-d) | (12.85) | (12.1%) | (9.8%) | (8.3%) | (10.3%) | (10.8%) |
| Specific | 1.0 | 0.92 | 1.04 | 0.95 | 0.94 | 0.93 |
| Productivity | (13.9%) | (19.6%) | (18.5%) | (15.5%) | (11.3%) | (15.4%) |

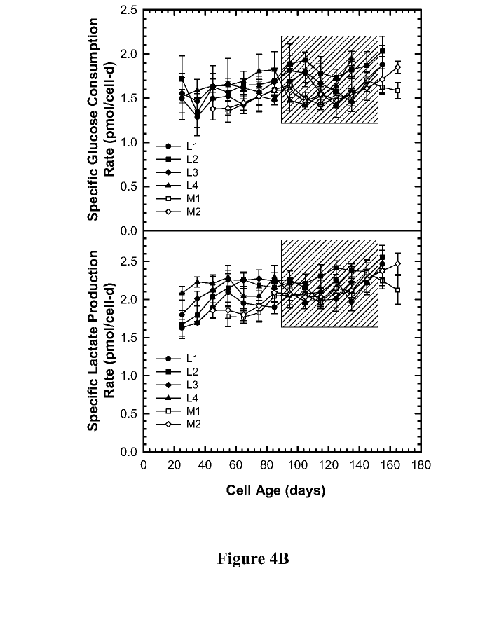

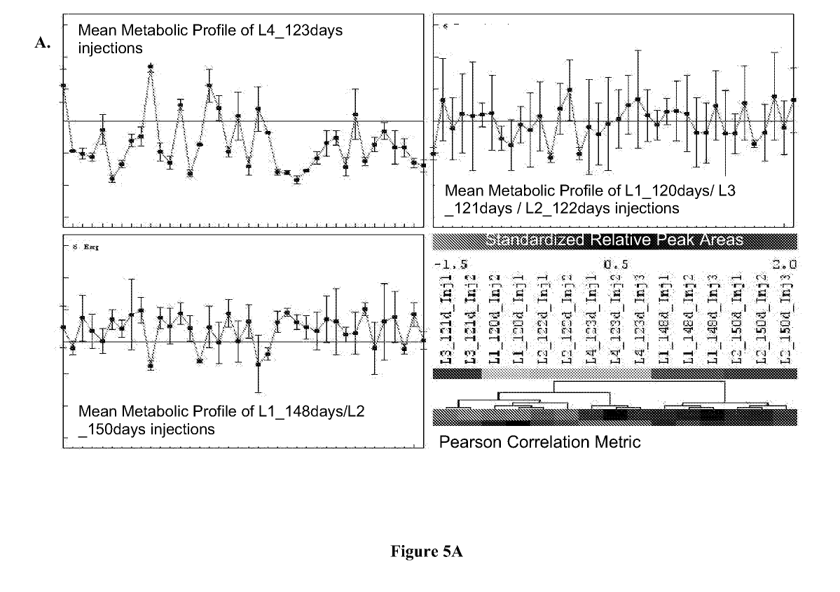

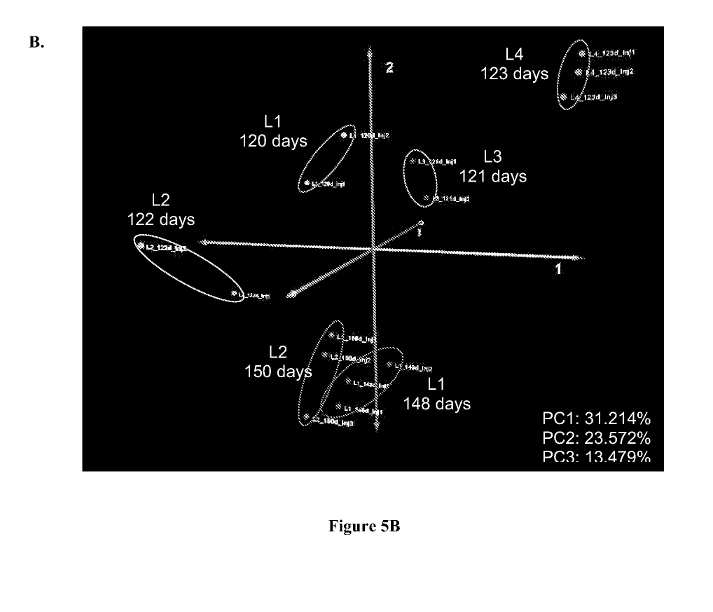

FIG. 4 provides the time profiles of a) bioreactor viability, b) cell growth, c) specific glucose consumption rate, and d) specific lactate production rate for the reactors in this study. The course of the cultivation for each reactor was divided into 10-days intervals starting from the time point at which steady-state was reached. The average value for each interval along and the associated standard deviation are presented at the middle time-point of the interval. Data from cell age of 20-30 days have been averaged and shown as corresponding to a cell age of 25 days. No significant time-related variation was seen for viability and growth rate. Slightly increasing trends were seen for the time profiles of specific glucose consumption and lactate production rates. There is no apparent difference in physiology due to the cell age in each of the reactors based on the set of measurements that were used in cell culture engineering for the monitoring of the process. Consistency among the reactors was also observed in the average specific protein productivity data (Table 2) where the productivity of the reactor L1 was arbitrarily set to 1.0 and used for comparison. The specific productivity range of 0.92-1.04 clearly indicates consistency among the bioreactors. In addition, material from the early, middle, and late stages from the bioreactors was purified and tested for typical protein quality attributes. The product quality data was within specifications suggesting consistency across bioreactors and over the course of the cultivation.

Data from the analysis of laboratory-scale bioreactors are shown in FIGS. 5A and 5B. A clear cell age based differentiation is seen on principal component 2 (FIG. 5B) where the metabolic profiles of the 120-123 day samples appear on the upper part of the graph, while those of the 148-150 day samples lie on the lower portion. The metabolic profiles of the same cell age cultures acquired from the different laboratory-scale reactors are clearly differentiated primarily on principal component 1. Because principal component 1 carries the largest proportion of variance in the original dataset, this differentiation indicates that metabolomic profiles could be discriminatory of vial to vial variability in the same cell bank and/or of cell culture growth in different bioreactors. This supports the ability of biomolecular fingerprinting to identify subtle differences in physiology that are not observable based on the current monitoring toolbox. Further examination of FIG. 5B indicates a separation of the L3 and L4 samples (on the right side of the graph) from the L1 and L2 samples of both investigated cell ages (on the left side of the graph).

The Hierarchical Clustering (HCL) analysis results shown in FIG. 5A confirm the visualized differences between the metabolic profiles in the PCA graph. Using the Pearson Correlation distance metric that clusters the metabolic profiles based on their shape, the obtained hierarchical clustering tree contains two major branches that correspond to the metabolic profiles of the two different cell ages. The Pearson correlation coefficient, r, between two metabolic profiles is equal to the covariance of the two profiles divided by the product of their standard deviations (Box et. al., 1978). It can take values between −1 and 1. The covariance is a measure of the linear dependence between two profiles. Thus, Pearson Correlation is expected to reveal similarities between the shapes of two profiles (Quackenbush, 2001).

Within the lower cell age branch (on the left of the tree), two separate subclusters were identified; one containing the metabolic profiles from the 3 injections of the 123 day L4 sample, with the metabolic profiles of the other three reactors' samples clustering in the other sub-branch. The L4 metabolic profile data points were further apart from the rest in the experimental space as shown from the PCA graph (FIG. 5B). The mean metabolic profile of the L4 injections shown in FIG. 5A (this centroid profile was generated from the TM4 MeV software; Saeed, et. al., 2003) differs from the mean metabolic profile of the rest of the reactors at similar cell age.

HCL analysis using Euclidean distance demonstrated the differentiation of the L3 and L4 profiles from the L1 and L2 profiles of both cell ages. Euclidean Distance Metric is the classic (most direct) distance between two points in the Euclidean space, which is defined by the Pythagorean theorem. In a M-dimensional space, the Euclidean distance between the points x=(x1, x2, . . . , xM) and y =(y1, y2, . . . , yM) is defined as:

$$\sqrt{{\sum }_{i = 1}^{M} {( {x}_{i} - {y}_{i} )}^{2}}$$

Thus, in a 2-dimensional space, the region enclosed by the points of the same Euclidean distance from an origin is a circle with radius equal to the particular distance.

Both PCA and HCL analyses indicated vial to vial variability and cell age to have higher impact on the measured polar metabolic profiles of cell culture samples than the reactor geometry (standard cylindrical versus small bottom configuration). With all these three parameters varying in the available laboratory-scale culture samples, the effect of reactor geometry on the metabolic physiology is not directly apparent in the clustering of the samples.

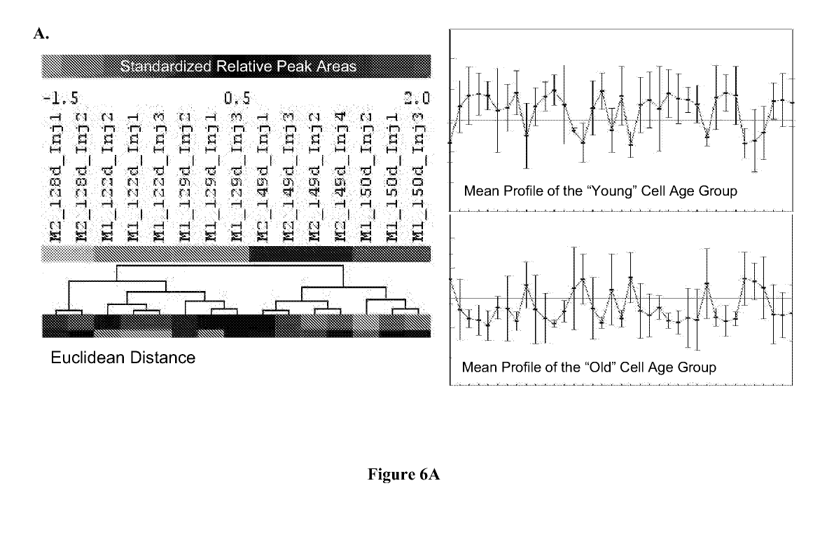

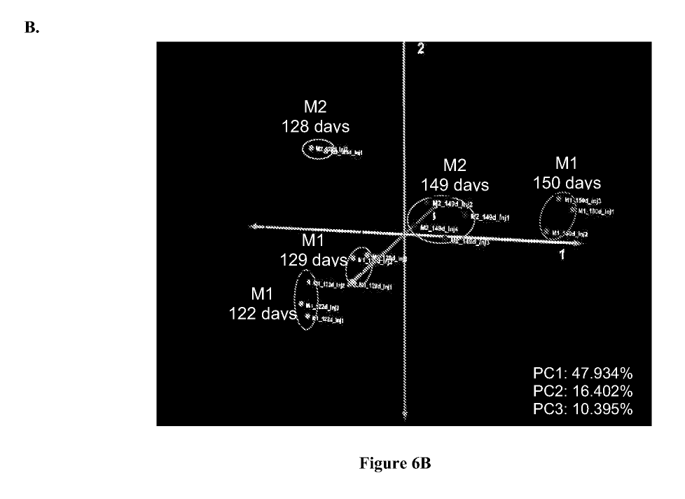

FIGS. 6A and 6B show the results of the HCL and PCA analyses, respectively, for the metabolic profiles of the manufacturing-scale reactor samples. Cell age based separation of samples is seen on principal component 1 (FIG. 6B). This separation is also apparent by HCL analysis (FIG. 6A) where the two main branches of the hierarchical clustering tree correspond to the two cell ages. The mean metabolic profiles of the two cell ages shown in FIG. 6A also support this differentiation.

Within the 122-129 day and 149-150 day sample groups, both HCL and PCA indicated clear differentiation between M1 and M2 samples. The difference between the M1 and M2 metabolic profiles of the 149-150 day samples was larger than the corresponding one in the 122-129 days old group. In the first case, samples were separated on principal component 1 (48% variance), while the second set of samples were separated on principal component 2 (16.5% variance).

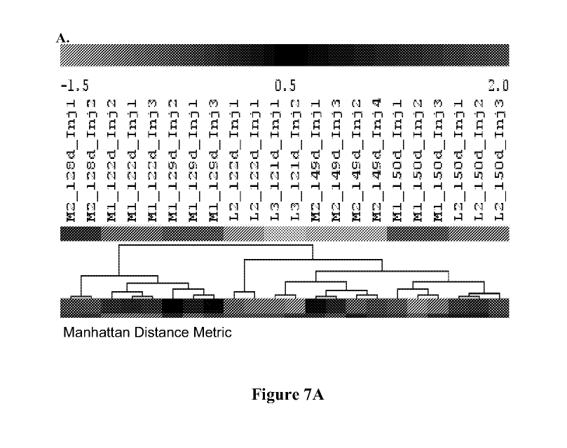

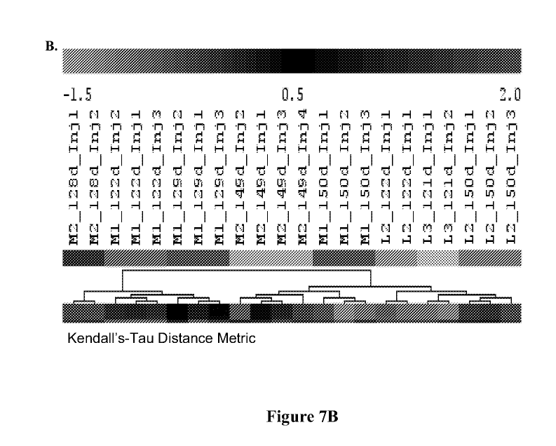

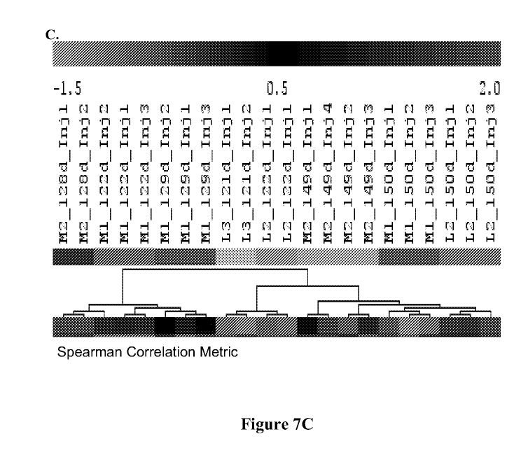

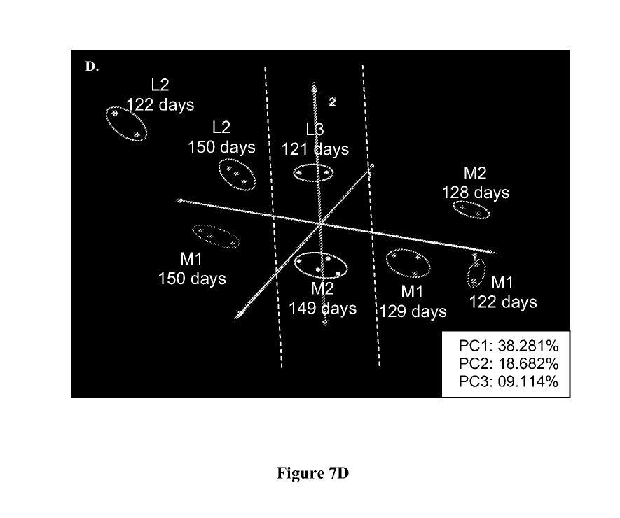

The 38 polar metabolite profiles of the manufacturing-scale bioreactors were subsequently analyzed in combination with the profiles of the laboratory-scale bioreactors from which they had been inoculated. FIGS. 7A-D show the results of the HCL and PCA analysis for this set of samples. The PCA and HCL analyses based on any of the distance metrics identified the metabolic profiles of the 122-129 days old samples of the manufacturing-scale bioreactors, M1 and M2, as a separate cluster from all other samples. In any of the HCL analyses (FIG. 7A-C), these samples form one of the two major branches of the trees, while they are also shown separated on the right side of the PCA graph (FIG. 7D).

The remaining five samples in FIG. 7 could be categorized based on cell age, reactor type, and cell source. Two samples (L2 and L3) had cell ages in the 121-122 day range while L2, M1, and M2 were 149-150 day samples. Three samples were from laboratory-scale bioreactors while two were from manufacturing-scale systems. Sample pairs L2-M1 and L3-M2 were from the same cell source (vials B and C, respectively). In FIG. 7D, these samples are clearly differentiated based on reactor size and cell source. Reactor size based differentiation was on principal component 2 where laboratory-scale reactors were on the positive side and the manufacturing-scale reactors on the negative side. Cell source based differentiation was on principal component 1 where the L2 and M1 samples clustered towards the left of the L3 and M2 samples.

The cell source based clustering is also apparent in the HCL analysis when Euclidean or Manhattan distance metric is used. In a M-dimensional space, the Manhattan distance between the points x =(x1, x2, . . . , xM) and y =(yl, y2, . . . , yM) is defined as the sum of the lengths of the projections of the (x,y) line segment onto the coordinate axes:

$${\sum }_{i = 1}^{M} \vert {x}_{i} - {y}_{i} \vert$$

In a 2-dimensional space, the region enclosed by the points of the same Manhattan distance from an origin is a square with sides oriented at a 45° angle to the coordinate axes. The Manhattan distance is less sensitive to outliers than the Euclidean distance (Filzmoser, et. al., Comput. Stat. Data Anal. 52:1694-1711, 2008). Both Manhattan and Euclidean distances measure absolute differences between the available data vectors (in this case the metabolic profiles). If used in clustering analysis, both metrics are expected to reveal similarities between the peak area levels of the metabolic profiles.

In FIG. 7A, the right branch of the hierarchical tree is divided into three branches, one clustering the M2 and L3 samples, the other the 150 day L2 and M1 samples, and the third the 122 day L2 sample, similar to the PCA graph (FIG. 7D). The clustering of metabolic profiles with respect to the reactor size in the HCL analysis was seen using the Kendall's Tau Distance Metric. This metric refers to the ranking vectors of the metabolic profiles. In the ranking vector of a metabolic profile, each relative peak area is replaced by the integer that indicates its ranking among all relative peak areas in the metabolic profile. In this case, the distance between two ranking vectors is measured based on the number of times the two integers are in opposite order in the two vectors. If this number is equal to zero, the two ranking vectors are identical.

In FIG. 7B, the right branch of the hierarchical tree is divided into two sub-branches corresponding to the laboratory-scale and the manufacturing-scale samples. Finally, HCL analysis using the Spearman Correlation distance metric illustrated sample clustering based on cell age. This correlation refers to the application of the Pearson Correlation Distance Metric on the ranking vectors of the metabolic profiles. In FIG. 7C, the right branch of the hierarchical tree is divided into two sub-branches, one containing the 121-122 day L2 and L3 samples and the other containing the 149-150 day L2, M1 and M2 samples.

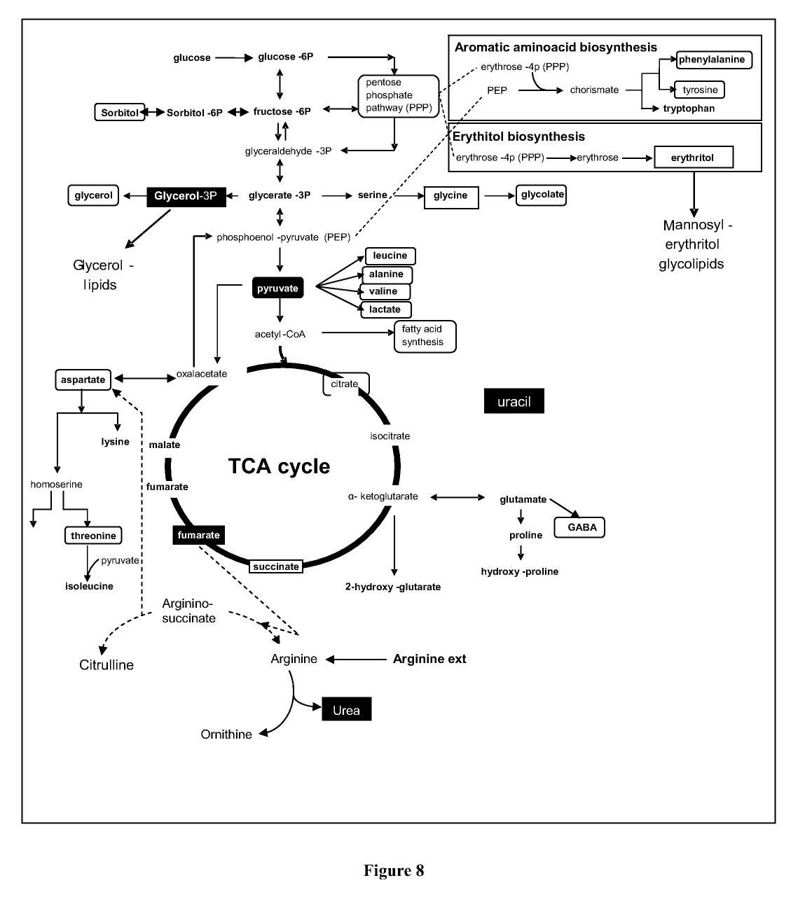

Significance Analysis for Microarrays (SAM) was used to identify the metabolites whose concentration significantly increased (positively significant) or decreased (negatively significant) in the 129 day M1 sample compared to the 122 day sample. Despite these samples being only a week apart, they could be differentiated from HCL and PCA analyses. SAM (for a delta value of 1.64 and 0% False Discovery Rate (FDR)) identified five positively and no negatively significant metabolites, which in order of decreasing significance were fumarate, glycerol-3-phosphate, urea, uracil, and pyruvate. Four metabolites are shown in FIG. 8 in the context of the metabolic network. The high-concentration of fumarate and urea may indicate a higher activity of the urea cycle and thus, of nitrogen assimilation in the 129 day compared to the 122 day sample. In addition, increased concentration of glycerol-3-phosphate may indicate increased production of glycerolipids. Uracil, which is the precursor of uridine and the main component of uridine phosphates (UMP, UDP and UTP), plays an important role in carbohydrate metabolism, protein glycosylation and glycolipid formation. It is apparent that this type of information can be useful in optimizing bioreactor operation.

Higher resolution characterization of cell physiological state was possible through metabolite profiling. Metabolomic profiles could be used to identify one or more discriminatory metabolites for a parameter of interest such as cell age. For example, metabolomic profiles could be used to identify one, two, five, ten, fifty, one hundred, or more discriminatory metabolites for a parameter of interest. These discriminatory metabolites could be used in combination with the conventionally measured cell culture physiological variables to optimize bioreactor cultivation and to serve as early warnings or process upsets. Metabolomics may be utilized as a sensitive high-throughput molecular analysis tool in cell culture engineering.

Other embodiments of the invention will be apparent to the skilled in the art from a consideration of this specification or practice of the invention disclosed herein. It is intended that the specification and examples be considered as exemplary only, with the true scope and spirit of the invention being indicated by the following claims.

1. A method for monitoring the physiological state of a cell culture, comprising the steps of:
(a) determining the level of one or more metabolites in a first culture sample taken from a first bioreactor;
(b) determining the level of one or more metabolites in at least a second culture sample taken from a second bioreactor; and
(c) comparing the level of one or more metabolites in a first culture sample with the level of metabolites in the second culture sample;
wherein a change in the level of one or more metabolites in the first culture sample compared to the level of one or more metabolites in the second culture sample is an indicator of the physiological state of a cell culture.

2. The method of claim 1, wherein the physiological state is selected from cell growth, metabolic profile, and cell age.

3. The method of claim 1, wherein the cell culture is a mammalian cell culture.

4. The method of claim 3, wherein the mammalian cell culture is a baby hamster kidney cell culture.

5. The method of claim 1, where in the bioreactor is selected from laboratory scale bioreactor, manufacturing scale bioreactor, perfusion bioreactor, and fed-batch bioreactor

6. The method of claim 1, wherein the levels of metabolites is determined by mass spectrometry and NMR.

7. The method of claim 1, wherein the levels of metabolites is determined by gas chromatography-mass spectrometry and liquid chromatography-mass spectrometry.

8. The method of claim 1, wherein the comparison of the levels of metabolites is determined using multivariate statistical analysis.

9. The method of claim 1, wherein the comparison of the levels of metabolites is determined using the method selected from hierarchial clustering and principal component analysis

10. The method of claim 1, wherein the metabolites are selected from amine group containing metabolites and ketone group containing metabolites.

11. The method of claim 1, wherein the metabolites are selected from glucose, lactate, glutamine, glutamate, ammonia, fumarate, glycerol-3-phosphate, urea, uracil, and pyruvate.

12. The method of claim 1, wherein the method is a high-throughput method.

13. The method of claim 1, wherein the metabolites are intracellular or extracellular.

14. A method for monitoring the physiological state of a cell culture, comprising the steps of:
(a) determining the level of one or more metabolites in a culture sample taken from a bioreactor; and
(b) comparing the level of one or more metabolites in the culture sample with the level of metabolites in a standard culture sample;
wherein a change in the level of one or more metabolites in the culture sample compared to the level of one or more metabolites in the standard culture sample is an indicator of the physiological state of a cell culture.

15. The method of claim 14, wherein the physiological state is selected from cell growth, metabolic profile, and cell age.

16. The method of claim 14, wherein the cell culture is a mammalian cell culture.

17. The method of claim 16, wherein the mammalian cell culture is a baby hamster kidney cell culture.

18. The method of claim 14, where in the bioreactor is selected from laboratory scale bioreactor, manufacturing scale bioreactor, perfusion bioreactor, and fed-batch bioreactor

19. The method of claim 14, wherein the levels of metabolites is determined by mass spectrometry and NMR.

20. The method of claim 14, wherein the levels of metabolites is determined by gas chromatography-mass spectrometry and liquid chromatography-mass spectrometry.

21. The method of claim 14, wherein the comparison of the levels of metabolites is determined using multivariate statistical analysis.

22. The method of claim 14, wherein the comparison of the levels of metabolites is determined using the method selected from hierarchial clustering and principal component analysis

23. The method of claim 14, wherein the metabolites are selected from amine group containing metabolites and ketone group containing metabolites.

24. The method of claim 14, wherein the metabolites are selected from glucose, lactate, glutamine, glutamate, ammonia, fumarate, glycerol-3-phosphate, urea, uracil, and pyruvate.

25. The method of claim 14, wherein the method is a high-throughput method.

26. The method of claim 14, wherein the metabolites are intracellular or extracellular.

27. A metabolomic profile generated from the method of claim 1 or claim 14.
